(19) United States
(12) Patent Application Publication
(10) Pub. No.: US 2016/0199359 A1
(43) Pub. Date: Jul. 14, 2016

(54) SYSTEM FOR TRANSDERMAL TREATMENT OF EMESIS

(71) Applicant: Genovate Biotechnology Co., Ltd., Hsin-Chu (TW)

(72) Inventor: Jen Chen, Hsin-Chu (TW)

(21) Appl. No.: 14/956,988

(22) Filed: Dec. 2, 2015

Related U.S. Application Data
(60) Provisional application No. 62/101,678, filed on Jan. 9, 2015.

(51) Int. Cl. A61K 31/439; A61K 9/70

(52) U.S. Cl. CPC A61K 31/439; A61K 9/7061

(57) ABSTRACT

An antiemetic system for weekly application that contains two identical transdermal patches, a first patch and a second patch, the first patch to be administered to a subject for 3 days followed by administering the second patch to the subject for 4 days. Also disclosed is a method of treating an emesis in a subject with the above-described antiemetic system.

### CROSS REFERENCE TO RELATED APPLICATIONS

This application claims priority to U.S. Provisional Application No. 62/101,678, filed on Jan. 9, 2015. The contents of this prior application are hereby incorporated by reference herein in its entirety.

### BACKGROUND OF THE INVENTION

Emesis is the most common side-effect for a cancer patient undergoing treatment as chemotherapy triggers release of serotonin, which is a 5-HT₃ (5-hydroxytryptamine 3) receptor agonist known to induce vomiting.

Granisetron, a 5-HT₃ receptor antagonist having a strong antiemetic effect, is a drug for treating nausea and vomiting caused by chemotherapy. Granisetron can be administered orally, rectally, or intravenously. However, a transdermal patch, both convenient and providing a therapeutically effective range for a long period of time, has emerged as a route of choice for administering Granisetron.

Granisetron transdermal patches are commercially available. Yet, they either require a large skin-contacting area, causing high discomfort to a patient, or do not conveniently provide a therapeutically effective dose over an extended period, e.g., a week.

There is a clear need to develop an improved Granisetron transdermal patch that, upon administration, achieves prolonged efficacy while causing minimal skin contacting-related discomfort. Ideally, this Granisetron transdermal patch can be supplied in a way self-manageable by a patient, e.g., in a kit, thereby facilitating administration compliance.

### SUMMARY OF THE INVENTION

This invention provides a system that includes two identical Granisetron transdermal patches that can be sequentially used to sustain a therapeutically effective blood concentration over a week with minimal skin discomfort. As such, it can be administered weekly for treating an emesis.

One aspect of this invention relates to an antiemetic system for weekly application that contains two identical transdermal patches, a first patch and a second patch, each having a skin-contacting area of 20-70 cm² (preferably, 20-50 cm²), the first patch administered to a patient for 3 days followed by administering the second patch to the patient for 4 days. The antiemetic system provides a dose-proportional area under curve (AUC) of plasma Granisetron and, for every 10 cm² skin-contacting area, delivers 1-4 mg of Granisetron in 7 days with a daily AUC of plasma Granisetron at 26.3±4.0 ng*hr/ml for each of days 2 to 7 of the administration.

The first patch and the second patch each include a backing layer, a release liner, and a matrix layer disposed between the backing layer and the release liner.

The matrix layer contains Granisetron, a permeation enhancer, and an adhesive, which are respectively 3-6%, 1-10%, and 84-96% by weight of the matrix layer.

The permeation enhancer can be a C₂-C₂₀ aliphatic alcohol, a C₁₀-C₂₀ aliphatic carboxylic acid, a C₂-C₁₀ amide, a C₂-C₁₀ lactam, or a combination thereof.

The adhesive can be an insoluble pressure-sensitive adhesive including an acrylic-based polymer.

Preferably, the second patch is administered to a site different from that for the first patch.

Another aspect of this invention relates to a method of treating emesis in a patient with the above-mentioned antiemetic system. The method includes: (i) administering the first patch to the patient for 3 days and (ii) administering the second patch to the patient for 4 days, thereby bringing about not only a dose-proportional AUC of plasma Granisetron but also, for every 10 cm² skin-contacting area, delivery of 1-4 mg of Granisetron in 7 days with a daily AUC of plasma Granisetron at 26.3±4.0 ng*hr/ml for each of days 2 to 7 of the administration.

Examples of emesis include acute emesis, delayed emesis, and anticipatory emesis.

The details of the invention are set forth in the description below. Other features, objects, and advantages of the invention will be apparent from the description and from the claims.

### DETAILED DESCRIPTION

A transdermal patch achieves therapeutic effects by permeating medical active ingredients into a patient via the skin thereof. The blood circulation then distributes the medical active ingredients throughout the patient's whole body. As described above, an antiemetic system of the present invention contains two identical Granisetron transdermal patches to be applied sequentially to a patient. In pharmacokinetics (PK) studies, it exhibited an in vivo plasma profile superior to those of other currently available transdermal patches, namely, (1) a dose-proportional plasma concentration of Granisetron and (2) for every 10 cm² skin-contacting area, delivery of 1-4 mg of Granisetron in 7 days with a plasma concentration of Granisetron at 1100±160 pg/ml and a daily AUC of plasma Granisetron at 26.3±4.0 ng*hr/ml for each of days 2 to 7 of the administration.

The two identical Granisetron transdermal patches each include a matrix layer that contains Granisetron, a permeation enhancer, and an adhesive. The permeation enhancer can be a C₂-C₂₀ aliphatic alcohol, a C₂-C₁₀ amide, or a C₁₀-C₂₀ aliphatic carboxylic acid. Examples of the C₂-C₂₀ aliphatic alcohol includes a propylene glycol, a benzyl alcohol, an oleyl alcohol, and a combination thereof. Examples of the C₂-C₁₀ amide include a dimethylacetamide and an N-methyl-2-pyrrolidone. An example of the C₁₀-C₂₀ aliphatic carboxylic acid is an oleic acid.

The adhesive includes one selected from the group consisting of DURO-TAK 87-2516 manufactured by DURO-TAK (DURO-TAK®87-2516), DURO-TAK 87-2287 manufactured by DURO-TAK (DURO-TAK®87-2287), GELVA-737 manufactured by GELVA (GELVA®737), and GELVA-788 manufactured by GELVA (GELVA®788).

Described below are three specific examples of administering an antiemetic system of this invention. The antiemetic system included two identical Granisetron transdermal patches, each having a matrix layer that contained Granisetron (4%), a permeation enhancer (oleyl alcohol, 5%), and an acrylic adhesive (Dura-Talc 387-2516, 91%).

These three examples are to be construed as merely illustrative, and not limitative of the remainder of the disclosure in any way whatsoever.

Without further elaboration, it is believed that one of ordinary skill in the field can, based on the description herein, utilize the present invention to its fullest extent without undue experimentation.

### Example 1

### Treating an Emesis in Subjects with an Antiemetic System of this Invention and the Resulting PK Profiles

The antiemetic system used in this example included a first patch and a second patch, each of which had a skin-contacting area of 42 cm². As many as 31 healthy subjects were treated by the antiemetic system in a study to assess the PK profile thereof.

Each subject was administered with the first patch for 3 days followed by the second patch for additional 4 days, receiving an estimated dose of 14 mg Granisetron over the 7-day administration.

Blood samples from each subject were collected at 0, 12, 24, 36, 48, 60, 72, 84, 96; 108, 120, 132, 144, 156, 168, 174, 180, 204 and 252 hours post applying the first patch. Plasma concentrations of Granisetron were measured from the samples and the results are shown in Table 1 below, Calculated PK profiles are shown in Table 2 also below.

TABLE 1
Mean plasma concentration of Granisetron (pg/ml) over time in
subjects treated with an antiemetic system for weekly application

| | Test drug | | |
|---|---|---|---|
| Time (h) | Mean | SD | CV (%) |
| 0 | n.d. | n.d. | n.d. |
| 12 | 1077.11 | 1287.35 | 119.5 |
| 24 | 3935.83 | 3450.24 | 87.7 |
| 36 | 4538.42 | 3336.77 | 73.5 |
| 48 | 4634.87 | 3714.66 | 80.1 |
| 60 | 4147.93 | 3157.65 | 76.1 |
| 72 | 3800.76 | 3045.59 | 80.1 |
| 84 | 3779.51 | 2760.63 | 73.0 |
| 96 | 5669.23 | 4823.21 | 85.1 |
| 108 | 5887.21 | 4463.16 | 75.8 |
| 120 | 5906.90 | 5394.53 | 91.3 |
| 132 | 4653.49 | 4058.09 | 87.2 |
| 144 | 4126.29 | 3671.94 | 89.0 |
| 156 | 3553.07 | 3307.01 | 93.1 |
| 168 | 3248.96 | 2943.01 | 90.6 |
| 174 | 2848.86 | 2808.43 | 98.6 |
| 180 | 2267.82 | 2189.27 | 96.5 |
| 204 | 1222.04 | 1168.85 | 95.6 |
| 252 | 526.91 | 600.43 | 114.0 |

TABLE 2
PK parameters in subjects treated with an
antiemetic system for weekly application

| Parameters | Mean | SD | CV(%) |
|---|---|---|---|
| All subjects (N = 31) | | | |
| AUC₀₋ₜ (pg/mL * h) | 805584.4 | 632400.4 | 78.5 |
| AUC_{0-∞} (pg/mL * h) | 832504.6 | 659594.8 | 79.2 |
| Cₘₐₓ (pg/mL) | 7068.49 | 5408.19 | 76.5 |
| Tₘₐₓ (h) | 84.77 | 39.67 | 46.8 |
| MRT (h) | 107.22 | 15.94 | 14.9 |
| T_{1/2} (h) | 31.93 | 7.10 | 22.2 |

Daily PK data were calculated using trapezoidal rule and the results show that, for every 10 cm² skin-contacting area, the antiemetic system thus administered delivered Granisetron in such a way that brought about a plasma concentration of Granisetron at 1080±130 pg/ml and a daily AUC of plasma Granisetron at 25.9±3.1 ng*hr/ml for each of days 2 to 7 of the administration.

### Example 2

### Treating an Emesis in Subjects with an Antiemetic System of this Invention and the Resulting PK Profiles

The antiemetic system used in this example included a first patch and a second patch, each having a skin-contacting area of 25 cm². 6 healthy subjects were treated by the antiemetic system in a study to assess the PK profile thereof. Each subject was administered with the first patch for 3 days followed by the second patch for additional 4 days, receiving an estimated dose of 8.3 mg Granisetron over the 7-day administration.

Blood samples from each subject were collected at 0, 12, 24, 36, 48, 60, 72, 84, 96; 108, 120, 132, 144, 156, 168, 174, 180, 204 and 252 hours post applying the first patch.

Plasma concentrations of Granisetron were subsequently measured from the samples. The results are shown in Table 3 below. Calculated PK profiles are shown in Table 4 also below.

TABLE 3
Plasma concentrations of Granisetron (pg/ml) over time in six subjects treated with an
antiemetic system for weekly application

| | Subject | | | | | | | | |
|---|---|---|---|---|---|---|---|---|---|
| Period | II | I | I | II | II | I | | | |
| Time (h) | 101 | 102 | 103 | 104 | 105 | 106 | T-Mean | SD | CV (%) |
| Test drug | | | | | | | | | |
| 0 | 141.51 | blq | blq | blq | blq | blq | 23.59 | 57.77 | 244.9 |
| 12 | 623.60 | 3056.22 | 329.62 | 576.34 | 1729.23 | 109.18 | 1070.70 | 1121.96 | 104.8 |
| 24 | 2639.05 | 7157.43 | 1622.90 | 2284.48 | 2622.95 | 677.34 | 2834.03 | 2244.75 | 79.2 |
| 36 | 2719.21 | 7612.69 | 2810.99 | 1509.23 | 1774.99 | 1976.35 | 3067.24 | 2286.27 | 74.5 |
| 48 | 3175.19 | 7229.55 | 2305.45 | 3421.94 | 957.19 | 2319.31 | 3234.77 | 2139.44 | 66.1 |
| 60 | 2609.93 | 5623.61 | 2087.87 | 2510.91 | 567.61 | 2293.41 | 2615.56 | 1651.40 | 63.1 |
| 72 | 1712.32 | 5254.21 | 1439.38 | 2299.24 | 404.95 | 1556.78 | 2111.15 | 1657.89 | 78.5 |
| 84 | 1065.52 | 7887.37 | 1173.17 | 1778.85 | 1173.44 | 1146.44 | 2370.80 | 2714.91 | 114.5 |
| 96 | 2340.08 | 10754.78 | 1891.26 | 2942.55 | 2887.93 | 1900.45 | 3786.18 | 3444.35 | 91.0 |
| 108 | 2839.96 | 8887.58 | 1756.17 | 3051.69 | 2991.91 | 3076.76 | 3767.35 | 2557.81 | 67.9 |
| 120 | 2685.86 | 8067.78 | 756.92 | 3247.63 | 1961.40 | 2131.87 | 3141.91 | 2552.85 | 81.3 |
| 132 | 2132.96 | 6102.50 | 757.46 | 2832.73 | 1431.91 | 2414.97 | 2612.09 | 1861.86 | 71.3 |
| 144 | 1453.21 | 5508.24 | 572.07 | 2324.02 | 903.77 | 1826.37 | 2097.95 | 1784.66 | 85.1 |
| 156 | 1494.00 | 4455.81 | 581.43 | 2052.21 | 641.47 | 1752.95 | 1829.65 | 1416.66 | 77.4 |
| 168 | 1514.27 | 3435.08 | 524.82 | 2220.68 | 745.79 | 1477.41 | 1653.01 | 1062.45 | 64.3 |
| 174 | 1400.18 | 3602.19 | 522.05 | 1203.77 | 609.61 | 1389.12 | 1454.49 | 1119.46 | 77.0 |
| 180 | 1298.94 | 2759.16 | 633.82 | 1248.37 | 437.01 | 1418.64 | 1299.32 | 816.63 | 62.9 |
| 204 | 548.52 | 1682.02 | 271.39 | 774.08 | 256.38 | 982.66 | 752.51 | 536.03 | 71.2 |
| 252 | 288.22 | 465.68 | 224.27 | 204.29 | 128.71 | 333.77 | 274.16 | 117.44 | 42.8 |

Concentration unit: pg/mL; blq means concentration below lower limit of quantification.

TABLE 4
PK parameters in subjects treated with an antiemetic system for weekly application

| | AUC₀₋ₜ | AUC_{0-∞} | AUC₀₋ₜ/ | Cₘₐₓ | Tₘₐₓ | MRT | T_{1/2} | |
|---|---|---|---|---|---|---|---|---|
| Parameters | (pg/mL * h) | (pg/mL * h) | AUC_{0-∞} | (pg/mL) | (h) | (h) | (h) | RSQ |
| Mean | 491377.6 | 504816.6 | 96.7 | 4371.93 | 74.00 | 108.04 | 35.34 | 0.9394 |
| SD | 363403.8 | 365523.7 | 1.9 | 3133.49 | 33.44 | 9.57 | 9.75 | 0.1015 |
| CV (%) | 74.0 | 72.4 | 1.9 | 71.7 | 45.2 | 8.9 | 27.6 | 10.8 |
| maximum | 1216818.5 | 1235033.1 | 98.5 | 10754.78 | 108.00 | 125.33 | 53.57 | 0.9963 |
| Minmum | 249531.8 | 266869.2 | 93.5 | 2810.99 | 36.00 | 97.66 | 27.11 | 0.7353 |
| Median | 381700.4 | 396809.8 | 97.1 | 3125.98 | 72.00 | 106.10 | 33.65 | 0.9766 |

Daily PK data were calculated using trapezoidal rule and the results show that, for every 10 cm² skin-contacting area, the antiemetic system thus administered delivered Granisetron in such a way that brought about a plasma concentration of Granisetron at 1110±160 pg/ml and a daily AUC of plasma Granisetron at 26.3±3.8 ng*hr/ml for each of days 2 to 7 of the administration.

### Example 3

### Treating an Emesis in Subjects with an Antiemetic System of this Invention and the Resulting PK Profiles

The antiemetic system used in this example included a first patch and a second patch, each having a skin-contacting area of 34 cm². 6 healthy subjects were treated by the antiemetic system in a study to assess the PK profile thereof. Each subject was administered at two different sites with the two first patches for 3 days followed by the two second patch for additional 4 days, receiving an estimated dose of 22.6 (11.3×2) mg Granisetron over the 7-day administration.

Blood samples from each subject were collected at 0, 12, 24, 36, 48, 60, 72, 84, 96; 108, 120, 132, 144, 156, 168, 174, 180, 204 and 252 hours post applying the first patch. Plasma concentrations of Granisetron were measured from the samples and the results are shown in Table 5 below. Calculated PK profiles are shown in Table 6 also below.

TABLE 5
Mean plasma concentration of Granisetron (pg/ml) over time
in subjects treated with an antiemetic system for weekly application

| | Test drug | | |
|---|---|---|---|
| Time (h) | Mean | SD | CV (%) |
| 0 | 14.56 | 35.65 | 244.9 |
| 12 | 4564.05 | 6420.43 | 140.7 |
| 24 | 9624.12 | 10750.98 | 111.7 |
| 36 | 9015.21 | 8631.46 | 95.7 |
| 48 | 7924.61 | 6479.34 | 81.8 |
| 60 | 6356.49 | 4631.23 | 72.9 |
| 72 | 5688.61 | 3937.49 | 69.2 |
| 84 | 6372.89 | 4201.21 | 65.9 |
| 96 | 8979.78 | 6525.38 | 72.7 |
| 108 | 10016.14 | 8865.76 | 88.5 |
| 120 | 7615.35 | 2038.55 | 26.8 |
| 132 | 7182.61 | 3104.39 | 43.2 |
| 144 | 7422.94 | 4424.67 | 59.6 |
| 156 | 6094.46 | 3779.49 | 62.0 |
| 168 | 5351.48 | 2599.80 | 48.6 |
| 174 | 4391.48 | 2335.04 | 53.2 |
| 180 | 4671.41 | 2009.50 | 43.0 |
| 204 | 2103.25 | 1265.27 | 60.2 |
| 252 | 624.63 | 390.75 | 62.6 |

Concentration unit: pg/mL

TABLE 6
PK parameters in subjects treated with an antiemetic system for weekly application

| | AUC₀₋ₜ | AUC_{0-∞} | AUC₀₋ₜ/ | Cₘₐₓ | Tₘₐₓ | MRT | T_{1/2} | |
|---|---|---|---|---|---|---|---|---|
| Parameters | (pg/mL * h) | (pg/mL * h) | AUC_{0-∞} | (pg/mL) | (h) | (h) | (h) | RSQ |
| Mean | 1397665.4 | 1421683.9 | 98.3 | 12176.51 | 86.00 | 107.46 | 24.31 | 0.9812 |
| SD | 919696.4 | 928034.3 | 1.3 | 9280.68 | 50.58 | 18.17 | 5.07 | 0.0079 |
| CV (%) | 65.8 | 65.3 | 1.3 | 76.20 | 58.8 | 16.9 | 20.8 | 0.8 |
| maximum | 3198548.5 | 3233190.3 | 99.5 | 30725.82 | 144.00 | 131.16 | 31.96 | 0.9964 |
| minmum | 624159.8 | 627562.9 | 96.1 | 6147.22 | 24.00 | 85.38 | 17.30 | 0.9749 |
| median | 1210302.7 | 1239269.3 | 98.4 | 8722.88 | 102.00 | 107.07 | 24.76 | 0.9790 |

Daily PK data were calculated using trapezoidal rule and the results show that, for every 10 cm² skin-contacting area, the antiemetic system thus administered delivered Granisetron in such a way that brought about a plasma concentration of Granisetron at 1110±150 pg/ml and a daily AUC of plasma Granisetron at 26.5±3.6 ng*hr/ml for each of days 2 to 7 of the administration.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. An antiemetic system for weekly application, comprising two identical transdermal patches, a first patch and a second patch, each of which has a skin-contacting area of 20-70 cm² and includes:
a backing layer,
a release liner, and
a matrix layer, disposed between the backing layer and the release liner, containing Granisetron 3-6% by weight of the matrix layer,
wherein the first patch is administered to a subject for 3 days followed by administering the second patch to the subject for 4 days, the system thereby providing a dose-proportional area under curve (AUC) of plasma Granisetron and, for every 10 cm² skin-contacting area, delivering 1-4 mg of Granisetron in 7 days with a daily AUC of plasma Granisetron at 26.3±4.0 ng*hr/ml for each of days 2 to 7 of the administration.

2. The antiemetic system of claim 1, wherein the system provides a dose-proportional plasma concentration of Granisetron and, for every 10 cm² skin-contacting area, delivers 1-4 mg of Granisetron in 7 days with a plasma concentration of Granisetron at 1100±160 pg/ml for each of days 2 to 7 of the administration.

3. The antiemetic system of claim 1, wherein the matrix layer further contains a permeation enhancer and an adhesive.

4. The antiemetic system of claim 3, wherein the permeation enhancer is 1-10% by weight of the matrix layer.

5. The antiemetic system of claim 4, wherein the permeation enhancer includes one selected from the group consisting of a C₂-C₂₀ aliphatic alcohol, a C₁₀-C₂₀ aliphatic carboxylic acid, a C₂-C₁₀ amide, a C₂-C₁₀ lactam, and a combination thereof.

6. The antiemetic system of claim 3, wherein the adhesive is 84-96% by weight of the matrix layer.

7. The antiemetic system of claim 6, wherein the adhesive is an insoluble pressure-sensitive adhesive including acrylic-based polymer.

8. The antiemetic system of claim 1, wherein the second patch is administered to a site different from that for the first patch.

9. A method of treating emesis in a subject with a system for weekly application that contains two identical transdermal patches, a first patch and a second patch, the method comprising:
administering the first patch to the subject for 3 days, and
administering the second patch to the subject for 4 days,
wherein the first patch and the second patch each have a skin-contacting area of 20-70 cm² and include:
a backing layer,
a release liner, and
a matrix layer, disposed between the backing layer and the release liner, containing Granisetron 3-6% by weight of the matrix layer,
whereby bringing about a dose-proportional area under curve (AUC) of plasma Granisetron and, for every 10 cm² skin-contacting area, delivery of 1-4 mg of Granisetron in 7 days with a daily AUC of plasma Granisetron at 26.3±4.0 ng*hr/ml for each of days 2 to 7 of the administration.

10. The method of claim 9, wherein the emesis is selected from a group consisting of acute emesis, delayed emesis, and anticipatory emesis.

11. The method of claim 9, wherein the system provides a dose-proportional plasma concentration of Granisetron and, for every 10 cm² skin-contacting area, delivers 1-4 mg of Granisetron in 7 days that sustains a plasma concentration of Granisetron at 1100±160 pg/ml for each of days 2 to 7 of the administration.

12. The method of claim 9, wherein the matrix layer further contains a permeation enhancer and an adhesive.

13. The method of claim 12, wherein the permeation enhancer is 1-10% by weight of the matrix layer.

14. The method of claim 13, wherein the permeation enhancer includes one selected from the group consisting of a C₂-C₂₀ aliphatic alcohol, a C₁₀-C₂₀ aliphatic carboxylic acid, a C₂-C₁₀ amide, a C₂-C₁₀ lactam, and a combination thereof.

15. The method of claim 12, wherein the adhesive is 84-96% by weight of the matrix layer.

16. The method of claim 15, wherein the adhesive is an insoluble pressure-sensitive adhesive including an acrylic-based polymer.

17. The method of claim 9, wherein the second patch is administered to a site different from that for the first patch.
